(19) United States
(12) Patent Application Publication
(10) Pub. No.: US 2026/0083748 A1
(43) Pub. Date: Mar. 26, 2026

(54) PHARMACEUTICAL COMPOSITION CONTAINING MELOXICAM, A PREPARATION METHOD, AND AN APPLICATION THEREOF

(71) Applicant: Beijing Tide Pharmaceutical Co., Ltd., Beijing (CN)

(72) Inventors: Wanjun LIU, Beijing (CN); Jingjing ZHANG, Beijing (CN); Zhibing ZHANG, Beijing (CN); Zaiying WANG, Beijing (CN); Di LU, Beijing (CN); Zhiyang YUE, Beijing (CN); Leling GAN, Beijing (CN)

(73) Assignee: Beijing Tide Pharmaceutical Co., Ltd., Beijing (CN)

(21) Appl. No.: 19/336,937

(22) Filed: Sep. 23, 2025

Related U.S. Application Data
(60) Provisional application No. 63/698,217, filed on Sep. 24, 2024.

(51) Int. Cl. A61K 31/5415; A61J 1/14; A61K 9/00; A61K 9/08; A61K 9/16; A61K 9/19; A61K 47/02; A61K 47/12; A61K 47/40; A61P 29/00

(52) U.S. Cl. CPC A61K 31/5415; A61J 1/1412; A61J 1/1468; A61K 9/0019; A61K 9/08; A61K 9/1611; A61K 9/1617; A61K 9/1652; A61K 9/19; A61K 47/02; A61K 47/12; A61K 47/40; A61P 29/00

(57) ABSTRACT

The present invention provides a pharmaceutical composition containing meloxicam, a preparation method thereof, and an application thereof. The pharmaceutical composition includes meloxicam, a composite solvent, and an acidic pH regulator. The composite solvent includes a polymer compound, an alkaline compound, and an aqueous solution. The polymer compound is selected from cyclodextrin or synthetic cyclodextrin and derivatives thereof. The meloxicam composition of the present invention does not contain any organic solvent, which can effectively avoid generation of adverse reactions of pharmaceutical formulations, make drug use safer, and at the same time also reduce the environmental pollution during the preparation process of the drug.

### CROSS-REFERENCE TO RELATED APPLICATIONS

This application claims the benefit of priority from US Provisional Application No. U.S. Provisional Application No. 63/698,217, filed Sep. 24, 2024, the contents of which are incorporated herein by reference.

### TECHNICAL FIELD

The present invention belongs to the field of pharmaceutical formulations and relates to a drug solution containing a poorly soluble non-steroidal anti-inflammatory drug, particularly relates to a drug solution containing meloxicam, its preparation method, and application.

### BACKGROUND ART

Meloxicam is a novel enolamides NSAID developed by BOEHRINGER INGELHEIM in Germany with unique pharmacological and pharmacodynamic effects. Its internal structure is very similar to that of its counterparts, such as piroxicam and tenoxicam, etc. Clinically, it is commonly used to treat osteoarthritis (OA), acute sciatica, rheumatoid arthritis (RA), etc. diseases. Since the incidence of gastrointestinal adverse reactions caused by oral administering meloxicam is much lower with than with other NSAIDs, currently meloxicam has been used as a substitute for other NSAIDs by many doctors in hospitals.

Meloxicam is a water insoluble weak acid compound with pH-dependent solubility. Its solubility is lowest at pH 4 and increases with the increase of pH value. Therefore, preparing meloxicam formulations requires an appropriate pH range to dissolve meloxicam, also ensuring solution stability during storage. These requirements have become a key and difficulty during preparing meloxicam solutions.

Patent publication CN031108024 discloses a meloxicam liquid formulation using cyclodextrin derivatives as solubilizers and stabilizers, by adding other excipients and a certain amount of buffer solutions to adjust pH value, the purpose of increasing the solubility of meloxicam is achieved. Patent publication CN201910390430.1 discloses a meloxicam injection for intravenous administration, which contains meloxicam, a cosolvent, and a pH regulator, wherein the cosolvent is a mixed solvent containing water and organic solvents, and can significantly enhances meloxicam solubility in liquid formulations and providing excellent stability.

In view of the above state art, it is necessary to develop new pharmaceutical compositions containing meloxicam, particularly those that can be used for intravenous injection.

### SUMMARY

The objective of the present invention is to provide a pharmaceutical composition containing meloxicam, especially a parenteral administration composition containing meloxicam, and in particular the present composition can be used for intravenous injection. The specific invention is as follows:

The present invention provides a pharmaceutical composition containing meloxicam, including meloxicam, a composite solvent, and an acidic pH regulator. The composite solvent comprises a polymer compound, an alkaline compound, and an aqueous solution. The polymer compound is selected from cyclodextrin or synthetic cyclodextrin and derivatives thereof.

As one of the embodiments of the present invention, the alkaline compound is selected from sodium hydroxide, sodium carbonate, sodium bicarbonate, sodium phosphate, disodium hydrogen phosphate, sodium dihydrogen phosphate, arginine, lysine, histidine, glycine, triethylamine, diethanolamine, or a combination of two or more thereof, preferably sodium hydroxide, sodium carbonate, or sodium bicarbonate, more preferably sodium hydroxide.

As one of the embodiments of the present invention, based on the volume of the composite solvent, the concentration of the alkaline compound is 0.01%-1% (w/v, g/mL), preferably 0.05%-0.5% (w/v, g/mL), more preferably 0.1%-0.2% (w/v, g/mL).

As one of the embodiments of the present invention, the polymer compound is selected from α-cyclodextrin, β-cyclodextrin, γ-cyclodextrin, hydroxypropyl-β-cyclodextrin, sulfobutyl-β-cyclodextrin sodium, or a combination of two or more thereof, preferably sulfobutyl-β-cyclodextrin sodium.

As one of the embodiments of the present invention, based on the volume of the composite solvent, the concentration of the polymer compound is 5%-40% (w/v, g/mL), preferably 8%-20% (w/v, g/mL), more preferably 10%-15% (w/v, g/mL).

The addition of sulfobutyl-β-cyclodextrin sodium in the formulation achieve significantly better the related substances profiles during stability sample placement process compared to formulations containing other cyclodextrins.

When the prescription does not contain cyclodextrin, the drug will be precipitated and the solution will become cloudy during the stable storage. Increasing the amount of cyclodextrin can enhance the solubilization effect and increase the drug content in the solution. When the cyclodextrin concentration of the solution is 10%-15% (w/v, g/mL), the drug concentration of the solution can reach 5-10 mg/mL. When the cyclodextrin concentration of the solution is 20%-40% (w/v, g/mL), the drug concentration of the solution can reach 10-15 mg/mL. The order of cyclodextrin addition affects the dissolution of meloxicam: adding the alkaline compound and meloxicam first, followed by cyclodextrin, results in faster dissolution, whereas adding cyclodextrin and the drug first, followed by the alkaline compound, results in prolonged dissolution time and reduced production efficiency.

As one of the embodiments of the present invention, the acidic pH regulator is an acidic aqueous solution. The acidic pH regulator is selected from hydrochloric acid, phosphoric acid, acetic acid, lactic acid, malic acid, fumaric acid, tartaric acid, succinic acid, citric acid, or a combination of two or more thereof, preferably citric acid. As an exemplary illustration, the formulations using citric acid as the pH regulator exhibit significantly better related substances profiles during stability sample placement process compared to those using other pH regulators.

As one embodiment of the present invention, based on the volume of the composite solvent, the concentration of the acidic pH regulator is 0.01%-1% (w/v, g/mL), preferably 0.05%-0.5% (w/v, g/mL), more preferably 0.08%-0.15% (w/v, g/mL).

As one of the embodiments of the present invention, the pH of the pharmaceutical composition is 6-10, preferably 7-9, more preferably 8-9.

Meloxicam is a non-steroidal anti-inflammatory drug (NSAID). Research has shown that NSAIDs act as COX-1 and COX-2 inhibitors. The inventors selected an alkaline aqueous solution containing polymer compounds as the solubilizer in the present invention, which significantly improves the solubility of meloxicam in alkaline solutions. The enhanced solubility of meloxicam further increases the bioavailability of the drug and greatly improves its therapeutic efficacy. The use of alkaline solubilizers and pH regulators in the present invention can maintain the stability while improving the solubility of meloxicam, effectively optimize the formulation and simplify the preparation method, so that the meloxicam solutions can be prepared at room-temperature under stirring conditions, and facilitate industrial-scale production.

As one of the embodiments of the present invention, the water in the pharmaceutical composition is selected from water for injection, distilled water, or purified water.

As one of the embodiments of the present invention, the drug concentration of meloxicam in the pharmaceutical composition is 1.0-15.0 mg/mL, preferably 2.0-10.0 mg/mL, more preferably 5.0-8.0 mg/mL.

As one of the embodiments of the present invention, the pharmaceutical composition further consists of:

| Meloxicam | 0.084 | parts |
|---|---|---|
| Sodium hydroxide | 0.020 | parts |
| Hydroxypropyl-β-cyclodextrin | 1.228 | parts |
| Hydrochloric acid | 0.015 | parts |
| Water for injection | 10 | parts; or |
| Meloxicam | 0.053 | parts |
| Sodium hydroxide | 0.014 | parts |
| β-cyclodextrin | 0.604 | parts |
| Acetic acid | 0.02 | parts |
| Water for injection | 10 | parts; or |
| Meloxicam | 0.082 | parts |
| Sodium hydroxide | 0.021 | parts |
| Sulfobutyl-β-cyclodextrin sodium | 1.232 | parts |
| Citric acid | 0.018 | parts |
| Water for injection | 9.568 | parts; or |
| Meloxicam | 0.23 | parts |
| Sodium hydroxide | 0.04 | parts |
| Sulfobutyl-β-cyclodextrin sodium | 3.982 | parts |
| Citric acid | 0.046 | parts |
| Water for injection | 10 | parts; or |
| Meloxicam | 0.076 | parts |
| Sodium hydroxide | 0.018 | parts |
| Sulfobutyl-β-cyclodextrin sodium | 1.634 | parts |
| Lactic acid | 0.018 | parts |
| Water for injection | 10 | parts; or |
| Meloxicam | 0.029 | parts |
| Sodium hydroxide | 0.014 | parts |
| Sodium carbonate | 0.018 | parts |
| β-cyclodextrin | 1.128 | parts |
| Phosphoric acid | 0.030 | parts |
| Lactic acid | Added as needed to pH 8.5 | |
| Water for injection | 9.568 | parts; or |
| Meloxicam | 0.015 | parts |
| Sodium hydroxide | 0.007 | parts |
| β-cyclodextrin | 0.522 | parts |
| Citric acid | 0.007 | parts |
| Water for injection | 10 | parts. |

As one of the embodiments of the present invention, the pharmaceutical composition is used for parenteral administration, preferably intravenous administration.

The present invention is primarily intended for intravenous administration. Compared to other administration routes, intravenous administration route is the fastest way to deliver the drug to the target sites, are also the most efficient and precise route. However, adverse reactions associated with this administration route may be harmful or cause discomfort to patients. Non-physiological osmolality, viscosity, or pH of drug solutions are the mechanisms that cause vascular inflammation and vascular tissue changes, resulting in discomfort in patients under such treatment. The present invention aims to reduce the side effects of meloxicam when administered intravenously, provided that the solubility of meloxicam is enhanced.

Pain and irritation at the injection site may be related to the formulation solvent, particularly those with high osmolality. The present invention does not use organic solvents, only use water as the solvent. Additionally, the formulation of this invention significantly reduces the pH dependence of the active ingredient, effectively minimizing pain and irritation during injection. Meanwhile, the excipients of the formulation of present invention is simple and safe, which greatly reduces the side effects of the formulation of the present invention.

As one of the embodiments of the present invention, the pharmaceutical composition is in the form of an injection or lyophilized powder, with a pH value of 6-9, preferably 7-9, more preferably 8-9.

As one of the embodiments of the present invention, the injection is used for intravenous administration, or the composition in the form of lyophilized powder is reconstituted for intravenous administration, wherein the reconstitution solvent is selected from water for injection, physiological saline, glucose, or other reconstitution solutions suitable for injection.

As one of embodiments of the present invention, the formulation in the form of lyophilized powder further includes a lyophilization stabilizer, the lyophilization stabilizer includes sucrose, lactose, maltose, glucose, raffinose, fructose, or dextrin.

As one of the embodiments of the present invention, the composition is sterilized, with no change in product weight after sterilization. The composition is sterilized by high-pressure sterilization or filtration sterilization, preferably high-pressure sterilization, wherein the sterilization temperature is 100° C.-150° C., more preferably 110° C.-130° C.

The formulation of the present invention may be sealed in inert gas. Various commercially available materials for aqueous liquid formulations that can be sterilized in the final containers can be used as packaging materials for the formulations of the present invention, which includes ampoules or vials, preferably vials, fitted with rubber stoppers and aluminum caps.

As one of the embodiments of the present invention, the aqueous liquid form of the composition is sealed in inert gas and sterilized in the final container, wherein the final container comprises ampoules or vials, preferably vials with rubber stoppers and aluminum caps.

The present invention further provides a method for preparing aforementioned pharmaceutical composition, the method comprises: firstly adding alkaline compound and meloxicam to water, then adding polymer compound; or first adding alkaline compound and polymer compound to water, then adding meloxicam; or adding meloxicam, alkaline compound, and polymer compound to water together. Among these methods, the method of firstly adding alkaline compound and polymer compound to water, then adding meloxicam, can achieves faster dissolution and better efficacy.

As one of the embodiments of the method of present invention, the method further includes: weighing the prescribed amounts of meloxicam, alkaline compound, and polymer compound, firstly adding the alkaline compound and polymer compound to water, then adding meloxicam, stirring to dissolve to form a clear solution, adjusting the pH to 6-9 with the acidic pH regulator, preferably to 7-9, more preferably 8-9, filling into vials, and sterilizing to obtain the meloxicam solution.

As one of the embodiments of the method of present invention, the polymer compound is selected from one or more of α-cyclodextrin, β-cyclodextrin, γ-cyclodextrin, hydroxypropyl-β-cyclodextrin, or sulfobutyl-β-cyclodextrin sodium, preferably sulfobutyl-β-cyclodextrin sodium.

The present invention further provides the use of the aforementioned pharmaceutical composition in the preparation of drugs for the treatment of pain, such as postoperative analgesia after abdominal surgeries, orthopedic surgeries, etc., as well as other types of moderate to severe pain in adults. In accordance with this aspect, there are provided methods for treating pain by administering an effective amount of the pharmaceutical compositions described herein to a patient in need thereof.

### Technical Effects

The focus of the present invention is to address issues related to the solubility and solution stability of poorly soluble drugs, and to characterize the efficacy through pharmacological studies. Pharmacodynamic comparisons using the Anjeso (meloxicam nanosuspension injection), a globally marketed product, as the control formulation showed that the formulation of the present invention has higher efficacy; or under the same efficacy, the formulation of the present invention exhibits lower drug exposure and higher safety. It provides a better option of postoperative analgesia treatment for patients suffered from moderate to severe pain.

The pharmaceutical composition containing meloxicam as described in the present invention, and the alkaline aqueous solution as described in the present invention can greatly achieve the effect of solubilizing meloxicam, and can effectively improve the stability of meloxicam, and reduce the dependence of meloxicam's solubility on pH, significantly reduce the irritation caused by meloxicam injection, improve patient compliance, and be more suitable for clinical intravenous injection.

Secondly, the present invention improves the stability of the drug solution during storage by adding cyclodextrin, keeping the medicinal solution in a clear state during storage. Furthermore, the addition of sodium sulfobutyl-β-cyclodextrin and citric acid can also improve the stability of the drug solution during storage, resulting in better results regarding related substances.

Thirdly, the pharmaceutical composition of the present invention contains fewer types of excipients and has a simple preparation process, which is prepared by stirring and dissolving at room temperature. This facilitates scaled-up production and can effectively reduce production costs. At the same time, by optimizing the order of adding materials during the preparation process, the dissolution of meloxicam can be accelerated and production efficiency can be improved.

Finally, compared to commercially available meloxicam nanosuspension injections administered by intravenous route, the present invention exhibits faster onset, better efficacy, and rapid pain relief. Unlike other meloxicam solutions by intravenous administration, the present invention contains no organic solvents and can effectively avoid the generation of adverse reactions of pharmaceutical formulations, enhancing drug safety, and at the same time reduce the environmental pollution during the drug preparation process.

### BRIEF DESCRIPTION OF THE DRAWINGS

FIG. 1a: Particle size of the formulation in Example 3;

FIG. 1b: Particle size of commercially available product Anjeso;

FIG. 2: Pharmacodynamic comparison study of meloxicam.

### DETAILED DESCRIPTION

The following examples were provided to further illustrate the present invention, not intended to limit the effective scope of the present invention in any way.

### Example 1

### Formulation:

Meloxicam 0.084 g
Sodium hydroxide 0.020 g
Hydroxypropyl-β-cyclodextrin 1.228 g
Hydrochloric acid 0.015 g
Water for injection 10 g.

Preparation method: The prescribed amounts of meloxicam, sodium hydroxide, and hydroxypropyl-β-cyclodextrin were weighed and dissolved in water to form a clear solution. The pH was adjusted to 8.6 with hydrochloric acid solution. The solution was filled into vials and sterilized to obtain the meloxicam solution. The drug concentration was measured to be 7.38 mg/mL.

### Example 2

### Formulation:

Meloxicam 0.053 g
Sodium hydroxide 0.014 g
β-cyclodextrin 0.604 g
Acetic acid 0.02 g
Water for injection 10 g.

Preparation method: prescribed amounts of meloxicam, sodium hydroxide, and β-cyclodextrin were weighed and dissolved in water to form a clear solution. The pH was adjusted to 8.3 with acetic acid solution. The solution was filled into vials and sterilized to obtain the meloxicam solution. The drug concentration was measured to be 4.56 mg/mL.

### Example 3

### Formulation:

Meloxicam 0.082 g
Sodium hydroxide 0.021 g
Sulfobutyl-β-cyclodextrin sodium 1.232 g
Citric acid 0.018 g
Water for injection 9.568 g

Preparation method: prescribed amounts of sodium hydroxide and sulfobutyl-β-cyclodextrin sodium were weighed and dissolved in water to form a clear solution. The prescribed amount of meloxicam was then added and stirred until dissolved. The pH was adjusted to 7.9 with citric acid solution. The solution was filled into vials and sterilized to obtain the meloxicam solution. The drug concentration was measured to be 8.55 mg/mL.

### Example 4

### Formulation:

Meloxicam 0.23 g
Sodium hydroxide 0.04 g
Sulfobutyl-β-cyclodextrin sodium 3.982 g
Citric acid 0.046 g
Water for injection 10 g.

Preparation method: prescribed amounts of sodium hydroxide and sulfobutyl-β-cyclodextrin sodium were weighed and dissolved in water to form a clear solution. The prescribed amount of meloxicam was then added and stirred until dissolved. The pH was adjusted to 8.1 with citric acid solution. The solution was filled into vials and sterilized to obtain the meloxicam solution. The drug concentration was measured to be 14.36 mg/mL.

### Example 5

### Formulation:

Meloxicam 0.076 g
Sodium hydroxide 0.018 g
Sulfobutyl-β-cyclodextrin sodium 1.634 g
Lactic acid 0.018 g
Water for injection 10 g

Preparation method: prescribed amounts of sodium hydroxide and sulfobutyl-β-cyclodextrin sodium were weighed and dissolved in water to form a clear solution. The prescribed amount of meloxicam was then added and stirred until dissolved. The pH was adjusted to 7.6 with lactic acid solution. The solution was filled into vials and sterilized to obtain the meloxicam solution. The drug concentration was measured to be 5.96 mg/mL.

### Example 6

### Formulation:

Meloxicam 0.029 g
Sodium hydroxide 0.014 g
Sodium carbonate 0.018 g
β-cyclodextrin 1.128 g
Phosphoric acid 0.030 g
Lactic acid added as needed to pH 8.5
Water for injection 9.568 g.

Preparation method: prescribed amount of meloxicam was dissolved in an aqueous solution of sodium hydroxide, sodium carbonate, and β-cyclodextrin. Other excipients were sequentially added to the solution according to the formulation. The pH was adjusted to 8.5 with 1M lactic acid. The solution was filled into vials and sterilized to obtain the meloxicam solution. The drug concentration was measured to be 3.01 mg/mL.

### Example 7

### Formulation:

Meloxicam 0.015 g
Sodium hydroxide 0.007 g
β-cyclodextrin 0.522 g
Citric acid 0.007 g
Water for injection 10 g.

Preparation method: prescribed amounts of meloxicam, sodium hydroxide, and β-cyclodextrin were weighed and dissolved in water to form a clear solution. The pH was adjusted to 7.9 with citric acid solution. The solution was filled into vials and sterilized to obtain the meloxicam solution. The drug concentration was measured to be 1.12 mg/mL.

### Comparative Example 1: Meloxicam Solution

### Formulation:

Meloxicam 0.050 g
Sodium hydroxide 0.020 g
Water for injection 9.546 g.

Preparation method: prescribed amounts of meloxicam and sodium hydroxide were weighed and dissolved in water for injection to form a clear solution. The solution was filled into vials to obtain the meloxicam solution. The drug concentration was measured to be 5.21 mg/mL.

The liquid medicine was an alkaline solution, and its irritancy was too strong, making it impossible to be directly used for injection.

### Comparative Example 2: Meloxicam Solution

### Formulation:

Meloxicam 0.057 g
Sodium hydroxide 0.020 g
Citric acid 0.019 g
Water for injection 9.539 g.

Preparation method: prescribed amounts of meloxicam and sodium hydroxide were weighed and dissolved in water for injection to form a clear solution. The pH was adjusted to 8.1 with citric acid solution. The solution was filled into vials to obtain the meloxicam solution. The drug concentration was measured to be 5.95 mg/mL.

The solution becomes slightly cloudy after stands for 72 hours, with drug precipitation.

### Comparative Example 3

Formulation:
Meloxicam 0.082 g
Sodium hydroxide 0.021 g
β-cyclodextrin 1.232 g
Citric acid 0.018 g
Water for injection 9.568 g.

Preparation method: prescribed amounts of meloxicam and β-cyclodextrin were weighed and stirred in water but fail to dissolve, forming a suspension. Sodium hydroxide was then added, gradually dissolving the mixture to form a clear solution. The pH was adjusted to 8.2 with citric acid solution. The solution was filled into vials and sterilized to obtain the meloxicam solution. The drug concentration was measured to be 8.3 mg/mL.

Compared with Example 2, dissolving meloxicam takes longer when cyclodextrin and the drug were added firstly, followed by sodium hydroxide.

In Examples 1, 2, 6, and 7, meloxicam, cyclodextrin, and sodium hydroxide were added to water together and stirred, completely dissolved for 18 minutes; whereas in Examples 3 to 5, cyclodextrin and sodium hydroxide were added to water firstly, followed by meloxicam, which was then stirred and completely dissolved, achieving complete dissolution over 10 minutes. The latter method results in shorter dissolution time and higher production efficiency.

### Comparative Example 4: Meloxicam Solution

### Formulation:

Meloxicam 0.068 g
Sodium carbonate 0.021 g
Lactic acid 0.019 g
Water for injection 9.385 g.

Preparation method: prescribed amounts of meloxicam and sodium carbonate were weighed and dissolved in water to form a clear solution. The pH was adjusted to 7.4 with lactic acid solution. The solution was filled into vials to obtain the meloxicam solution. The drug concentration was measured to be 7.21 mg/mL.

After standing for 72 hours, the solution becomes slightly cloudy with drug precipitation.

### Comparative Example 5

### Formulation:

Meloxicam 0.025 g
Glycine 0.022 g
Water for injection 4.915 g.

Preparation method: prescribed amounts of meloxicam and glycine were weighed and dissolved in water to form a solution. The solution was filled into vials and sterilized to obtain the meloxicam solution. The drug concentration was measured to be 5.08 mg/mL.

After standing for 72 hours, the solution becomes slightly cloudy with drug precipitation.

### Comparative Example 6

### Formulation:

Meloxicam 1.0 g
PEG400 15 mL
Sodium hydroxide/citric acid, As needed
Water for injection Up to 100 mL.

Preparation method: prescribed amounts of meloxicam and PEG400 were weighed and dissolved in water to form a solution. The pH was adjusted to 8.2 with sodium hydroxide/citric acid. The solution was filled into vials and sterilized to obtain the meloxicam solution. The drug concentration was measured to be 9.88 mg/mL.

### Experimental Example 1: Stability Test of Meloxicam Injection

A. Samples prepared in Examples 1, 2, 3, 5 and Comparative Examples 2, 3 were stored under conditions of a temperature of 25° C.±2° C. and a relative humidity of 60%±5% for 3, 6, and 12 months. The changes in drug concentration were measured, and the results were shown in Table 1.

TABLE 1
Stability Test of Meloxicam Injection (Drug Concentration)

| | | | | | | Drug | Drug |
|---|---|---|---|---|---|---|---|
| | | Drug | Drug | Drug | Drug | concentration in | concentration in |
| | | concentration | concentration | concentration | concentration | Comparative | Comparative |
| Storage | | in Example 1 | in Example 2 | in Example 3 | in Example 5 | Example 2 | Example 3 |
| Time | | (mg/mL) | (mg/mL) | (mg/mL) | (mg/mL) | (mg/mL) | (mg/mL) |
| 0 | h | 7.38 | 4.56 | 8.55 | 5.96 | 5.95 | 8.3 |
| 3 | months | 7.38 | 4.56 | 8.55 | 5.97 | Cloudy | 8.28 |
| | | | | | | solution | |
| 6 | months | 7.36 | 4.55 | 8.54 | 5.96 | NA | 8.27 |
| 12 | months | 7.36 | 4.56 | 8.53 | 5.95 | NA | 8.24 |

TABLE 2
Stability Test of Meloxicam Injection (Related Substances)

| | | | | | | Total | Total |
|---|---|---|---|---|---|---|---|
| | | Total | Total | Total | Total | Impurities in | Impurities in |
| | | Impurities in | Impurities in | Impurities in | Impurities in | Comparative | Comparative |
| Storage | | Example 1 | Example 2 | Example 3 | Example 5 | Example 2 | Example 3 |
| Time | | (%) | (%) | (%) | (%) | (%) | (%) |
| 0 | h | 0.10 | 0.08 | 0.04 | 0.05 | 0.05 | 0.04 |
| 3 | months | 0.72 | 0.62 | 0.20 | 0.32 | Cloudy | 0.23 |
| | | | | | | solution | |
| 6 | months | 1.02 | 0.98 | 0.29 | 0.51 | NA | 0.31 |
| 12 | months | 1.54 | 1.32 | 0.35 | 0.78 | NA | 0.38 |

As shown in Table 1, the drug concentration of the formulations in the present invention was significantly enhanced. After sterilization, the formulations maintain good drug concentration and demonstrates superior stability after the storage of long-term stability testing, which not only aids in drug storage, but also ensures medication safety. Formulations without cyclodextrin becomes cloudiness with drug precipitation during stability storage. Adding cyclodextrin and the drug firstly, followed by sodium hydroxide, which prolongs dissolution time and reduces production efficiency, while increasing the dosage of cyclodextrin can enhance the solubilization effect and increase the drug content in the solution.

During the preparation process of the above-mentioned Examples, it was unexpectedly discovered that the dissolution rate of Example 3 was fast and the drug solution was stable, indicating that the alkaline polymer solution can achieve rapid dissolution and stabilize the drug solution.

As shown in Table 2, compared with formulations containing other cyclodextrins, formulations containing sulfobutyl-β-cyclodextrin sodium exhibit significantly better results of related substances during stability sample placement process. Additionally, compared with formulations containing other acidic pH regulators, formulations using citric acid as the pH regulator show significantly better results of related substances.

B. Samples from Example 3, Comparative Example 6, and the control drug Anjeso were stored under the conditions of a temperature of 60° C.±2° C. for 0, 5, 10, and 30 days. Related substances were measured, and the results were shown in Table 3 below.

TABLE 3
High-Temperature Stability Test Results (Related Substances)

| | | Example 3 | | Comparative Example 6 | | control Drug | |
|---|---|---|---|---|---|---|---|
| | | Single | Total | Single | Total | Single | Total |
| Storage | | Impurity | Impurities | Impurity | Impurities | Impurity | Impurities |
| Time | | (%) | (%) | (%) | (%) | (%) | (%) |
| 0 | h | 0.03 | 0.04 | 0.03 | 0.14 | 0.01 | 0.10 |
| Day | 5 | 0.05 | 0.13 | 0.02 | 0.20 | NA | NA |
| Day | 10 | 0.06 | 0.20 | 0.05 | 0.27 | NA | NA |
| Day | 30 | 0.2 | 0.77 | 0.25 | 0.95 | 0.12 | 0.58 |

The results of the related substance examination indicate that the stability of the formulation solution of the present invention is good. Under high-temperature conditions, the rise in impurities is better than that of the formulation in the comparative example 6. Therefore, it can be inferred that the shelf life of this product is longer than that of the formulation in the comparative example 6.

### Experimental Example 2: Stability Test of Meloxicam Lyophilized Formulation

Samples from Examples 1, 2, 3, 5, and Comparative Example 3 were lyophilized for observation. The lyophilized products form uniform, loose cakes. They were stored under the conditions of a temperature of 25° C.±2° C. and a relative humidity of 45%+5% for 3, 6, and 12 months. The lyophilized products were reconstituted with water, and the drug concentrations were measured. The results were shown in Table 4.

TABLE 4
Stability Test of Lyophilized Meloxicam Products

| | | Post- | Post- | Post- | Post- | Post-lyophilization |
|---|---|---|---|---|---|---|
| | | lyophilization | lyophilization | lyophilization | lyophilization | content in |
| | | content in | content in | content in | content in | Comparative |
| Storage | | Example 1 | Example 2 | Example 3 | Example 5 | Example 3 |
| Time | | (mg/mL) | (mg/mL) | (mg/mL) | (mg/mL) | (mg/mL) |
| 0 | h | 7.38 | 4.56 | 8.55 | 5.96 | 8.3 |
| 3 | months | 7.36 | 4.54 | 8.52 | 5.92 | 8.26 |
| 6 | months | 7.36 | 4.55 | 8.54 | 5.93 | 8.26 |
| 12 | months | 7.35 | 4.53 | 8.53 | 5.94 | 8.25 |

The results in Table 4 show that the formulation of the present invention undergoes lyophilization processing, the product shape remains stable, the solubility of the product is good after reconstitution, no insoluble substances are generated, and the drug concentration remains basically unchanged. After stability testing, the results show that the concentration of the lyophilization product can maintain a good drug concentration and can be stably preserved.

### Experimental Example 3: Particle Size Comparison of Meloxicam

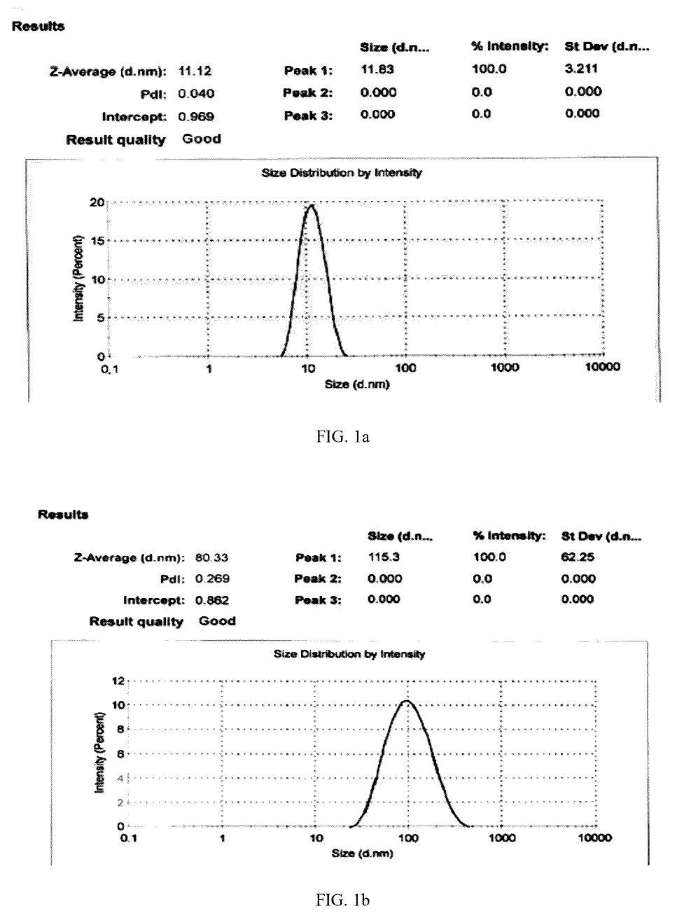

The particle sizes of the formulation in Example 3 and the commercially available product Anjeso were compared using a Malvern Nano Particle Size Analyzer. The results were shown in FIGS. 1a and 1b.

The results in FIGS. 1a and 1b indicate that compared with the commercially available product Anjeso, the particle size of the formulation in the present invention was significantly smaller, and the present formulation has faster absorption and onset of action after intravenous injection.

### Experimental Example 4: Efficacy Comparison of Meloxicam

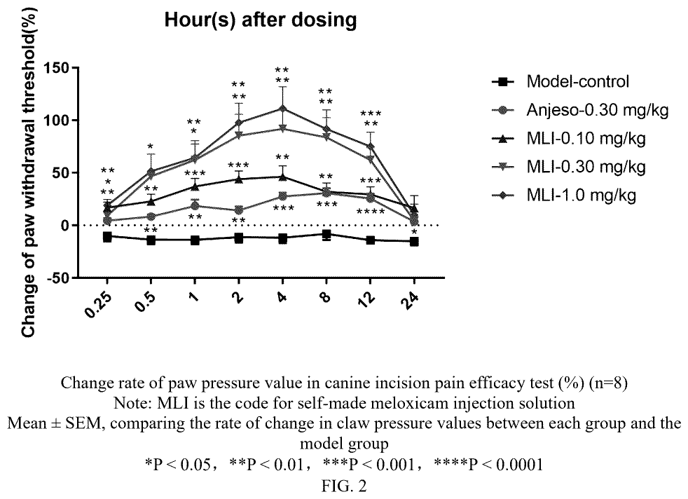

A comparative efficacy study was carried out on the samples prepared in Example 3 and the commercially available product Anjeso using Beagle dogs. Surgical incisions were made on the inner thighs of dogs to establish a pain model, reducing pain thresholds. On the second day post-surgery, the test samples and Anjeso were administered intravenously. The paw retraction pressure values of the animals were measured at 15 minutes, 30 minutes, 1 hour, 2 hours, 4 hours, 8 hours, 12 hours, and 24 hours before modeling, after modeling and before administration, after administration, respectively. The results were shown in FIG. 2. The results indicate that 0.1 mg/kg, 0.3 mg/kg, and 1 mg/kg of the self-made meloxicam injection can all increase the rate of change in the paw retraction pressure value of dogs in the incision pain model. The analgesic effect is dose-dependent and time-dependent, and it takes effect just at 0.25 hour after administration, reaches its peak at 4 hours, and the analgesic effect lasts until 12 hours. At the peak time point, the rate of change in the paw retraction pressure value shows a trend of the self-made product 1.0 mg/kg group>self-made product 0.3 mg/kg group>self-made product 0.1 mg/kg group>Anjeso group 0.3 mg/kg group>model group.

In conclusion, the results in FIG. 2 indicate that for the rate of change of paw retraction pressure values in beagle dogs after administration of the present invention formulation and the commercially available product Anjeso, the efficacy of the present invention formulation was significantly higher than that of Anjeso at all time points. This suggests that the present invention can have a faster onset of action and have a better therapeutic effect after administration. This may be related to the fact that this product can release drugs more quickly after entering the body, reach the site of action with the blood circulation, and thereby exert analgesic effects.

What is claimed is:

1. A pharmaceutical composition containing meloxicam, wherein the pharmaceutical composition comprises meloxicam, a composite solvent, and an acidic pH regulator, wherein the composite solvent comprises a polymer compound, an alkaline compound, and an aqueous solution, and the polymer compound is selected from cyclodextrin or synthetic cyclodextrin and derivatives thereof.

2. The pharmaceutical composition according to claim 1, wherein the alkaline compound is selected from sodium hydroxide, sodium carbonate, sodium bicarbonate, sodium phosphate, disodium hydrogen phosphate, sodium dihydrogen phosphate, arginine, lysine, histidine, glycine, triethylamine, diethanolamine, or a combination of two or more thereof, preferably sodium hydroxide, sodium carbonate, or sodium bicarbonate, more preferably sodium hydroxide.

3. The pharmaceutical composition according to claim 1, wherein, based on the volume of the composite solvent, the concentration of the alkaline compound is 0.01%-1% (w/v, g/mL), preferably 0.05%-0.5% (w/v, g/mL), more preferably 0.1%-0.2% (w/v, g/mL).

4. The pharmaceutical composition according to claim 1, wherein the polymer compound is selected from α-cyclodextrin, β-cyclodextrin, γ-cyclodextrin, hydroxypropyl-β-cyclodextrin, sulfobutyl-β-cyclodextrin sodium, or a combination of two or more thereof, preferably sulfobutyl-β-cyclodextrin sodium.

5. The pharmaceutical composition according to claim 1, wherein based on the volume of the composite solvent, the concentration of the polymer compound is 5%-40% (w/v, g/mL), preferably 8%-20% (w/v, g/mL), more preferably 10%-15% (w/v, g/mL).

6. The pharmaceutical composition according to claim 1, wherein the acidic pH regulator is an acidic aqueous solution, and the pH regulator is selected from hydrochloric acid, phosphoric acid, acetic acid, lactic acid, malic acid, fumaric acid, tartaric acid, succinic acid, citric acid, or a combination of two or more thereof, preferably citric acid.

7. The pharmaceutical composition according to claim 1, wherein based on the volume of the composite solvent, the concentration of the acidic pH regulator is 0.01%-1% (w/v, g/mL), preferably 0.05%-0.5% (w/v, g/mL), more preferably 0.08%-0.15% (w/v, g/mL).

8. The pharmaceutical composition according to claim 1, wherein the pH of the pharmaceutical composition is 6-10, preferably 7-9, more preferably 8-9.

9. The pharmaceutical composition according to claim 1, wherein the water in the pharmaceutical composition is selected from water for injection, distilled water, or purified water.

10. The pharmaceutical composition according to claim 1, wherein the drug concentration of meloxicam in the pharmaceutical composition is 1.0-15.0 mg/mL, preferably 2.0-10.0 mg/mL, more preferably 5.0-8.0 mg/mL.

11. The pharmaceutical composition according to claim 1, wherein the pharmaceutical composition further consists of:
Meloxicam 0.084 parts
Sodium hydroxide 0.020 parts
Hydroxypropyl-β-cyclodextrin 1.228 parts
Hydrochloric acid 0.015 parts
Water for injection 10 parts; or
Meloxicam 0.053 parts
Sodium hydroxide 0.014 parts
β-cyclodextrin 0.604 parts
Acetic acid 0.02 parts
Water for injection 10 parts; or
Meloxicam 0.082 parts
Sodium hydroxide 0.021 parts
Sulfobutyl-β-cyclodextrin sodium 1.232 parts
Citric acid 0.018 parts
Water for injection 9.568 parts; or
Meloxicam 0.23 parts
Sodium hydroxide 0.04 parts
Sulfobutyl-β-cyclodextrin sodium 3.982 parts
Citric acid 0.046 parts
Water for injection 10 parts; or
Meloxicam 0.076 parts
Sodium hydroxide 0.018 parts
Sulfobutyl-β-cyclodextrin sodium 1.634 parts
Lactic acid 0.018 parts
Water for injection 10 parts; or
Meloxicam 0.029 parts
Sodium hydroxide 0.014 parts
Sodium carbonate 0.018 parts
β-cyclodextrin 1.128 parts
Phosphoric acid 0.030 parts
Lactic acid Added as needed to pH 8.5
Water for injection 9.568 parts; or
Meloxicam 0.015 parts
Sodium hydroxide 0.007 parts
β-cyclodextrin 0.522 parts
Citric acid 0.007 parts
Water for injection 10 parts.

12. The pharmaceutical composition according to claim 1, wherein the pharmaceutical composition is used for parenteral administration, preferably intravenous administration.

13. The pharmaceutical composition according to claim 12, wherein the pharmaceutical composition is in the form of an injection or lyophilized powder, with a pH value of 6-9, preferably 7-9, more preferably 8-9.

14. The pharmaceutical composition according to claim 13, wherein the injection is used for intravenous administration; or the composition in the form of lyophilized powder is reconstituted for intravenous administration, wherein the reconstitution solvent is selected from water for injection, physiological saline, glucose, or other reconstitution solutions suitable for injection.

15. The pharmaceutical composition according to claim 13, wherein the composition in the form of lyophilized powder and further includes a lyophilization stabilizer, wherein the lyophilization stabilizer includes sucrose, lactose, maltose, glucose, raffinose, fructose, or dextrin.

16. The pharmaceutical composition according to claim 13, wherein the composition is sterilized by high-pressure sterilization or filtration sterilization, preferably high-pressure sterilization, wherein the sterilization temperature is 100° C.-150° C., preferably 110° C.-130° C.

17. The pharmaceutical composition according to claim 13, wherein the aqueous liquid form of the composition is sealed under inert gas and sterilized in the final container, wherein the final container comprises ampoules or vials, preferably vials fitted with rubber stoppers and aluminum caps.

18. A method for preparing the pharmaceutical composition according to claim 1, comprising: first adding alkaline compound and meloxicam to water, then adding polymer compound; or first adding alkaline compound and a polymer compound to water, then adding meloxicam; or adding meloxicam, an alkaline compound, and a polymer compound to water together; preferably, first adding alkaline compound and polymer compound first, then adding meloxicam.

19. The method according to claim 18, further comprising: weighing the prescribed amounts of meloxicam, alkaline compound, and polymer compound, first adding the alkaline compound and the polymer compound to water, then adding meloxicam, stirring to dissolve to form a clear solution, adjusting the pH to 6-9 with the pH regulator, preferably 7-9, more preferably 8-9, filling into vials, and sterilizing to obtain the meloxicam solution.

20. The method according to claim 19, wherein, the polymer compound is selected from one or more of α-cyclodextrin, β-cyclodextrin, γ-cyclodextrin, hydroxypropyl-β-cyclodextrin, or sulfobutyl-β-cyclodextrin sodium, preferably sulfobutyl-β-cyclodextrin sodium.

21. A method for treating pain by administering an effective amount of the pharmaceutical composition according to claim 1 to a patient in need thereof.

22. The method according to claim 21, wherein, the pharmaceutical composition is administered for postoperative analgesia and treatment of other types of moderate to severe pain in adults.

23. The method according to claim 22, wherein, the postoperative analgesia includes analgesia after abdominal surgery or orthopedic surgery.
